(19) United States
(12) Patent Application Publication
(10) Pub. No.: US 2025/0255591 A1
(43) Pub. Date: Aug. 14, 2025

(54) DEVICES, SYSTEMS, AND METHODS FOR MULTIPLE BIOPSY COLLECTION

(71) Applicant: BOSTON SCIENTIFIC MEDICAL DEVICE LIMITED, Galway (IE)

(72) Inventors: Nachiket GOLE, Pune (IN); Subodh MOREY, Ponda (IN); Samriddhi JAIN, Delhi (IN); Arun ADHIKARATH BALAN, Aluva (IN); Shrikant Vasant RAUT, Pune (IN); Nidhi DHINGRA, Gurgaon (IN)

(73) Assignee: BOSTON SCIENTIFIC MEDICAL DEVICE LIMITED, Galway (IE)

(21) Appl. No.: 19/051,475

(22) Filed: Feb. 12, 2025

Related U.S. Application Data
(60) Provisional application No. 63/552,723, filed on Feb. 13, 2024.

(51) Int. Cl. A61B 10/04; A61B 10/06

(52) U.S. Cl. CPC A61B 10/04; A61B 10/06

(57) ABSTRACT

Devices, systems and methods for multiple biopsy collection are described. The medical device may include a shaft defining a working channel extending from a proximal portion of the shaft to an opening at a distal tip of the shaft. A distalmost face of the distal tip may include an imaging device and a light source proximate the opening. The working channel may have a first size at the opening and a second size proximal to the imaging device and the light source, e.g., the second size being greater than the first size. At least a portion of the shaft around the opening may comprise an elastic material such that the opening is expandable in a direction away from the imaging device and the light source.

### CROSS-REFERENCE TO RELATED APPLICATIONS

This application claims the benefit of priority to U.S. Provisional Application No. 63/552,723, filed on Feb. 13, 2024, which is incorporated by reference herein in its entirety.

### TECHNICAL FIELD

Aspects of the present disclosure generally relate to medical devices, system, and procedures related thereto. In particular, some aspects relate to medical systems, devices, and methods for biopsy collection within the body of a patient.

### BACKGROUND

Medical scopes such as ureteroscopes and endoscopes typically feature electronic components and a working channel. In order to traverse through a patient's anatomy, the scopes often have a reduced profile which limits the number and types of instruments that may be passed through the working channel. Further, space at a distal face of the scope to accommodate electronic components such as imaging and illumination features is limited. Some medical procedures may require multiple instruments to be used over the duration of the medical procedure. Removal and replacement of instruments during medical procedures increases the duration of the procedure and may lead to various complications.

### SUMMARY

The present disclosure includes a medical device comprising a shaft defining a working channel extending from a proximal portion of the shaft to an opening at a distal tip of the shaft, e.g., the opening being expandable. A distalmost face of the distal tip may include an imaging device and a light source proximate the opening. The working channel may have a first size at the opening and a second size proximal to the imaging device and the light source that may be greater than the first size. At least a portion of the shaft around the opening may include an elastic material such the opening is expandable in a direction away from the imaging device and the light source. The elastic material may include a flexible, elastic polymer such as, e.g., silicone or polyurethane. A portion of the shaft opposite the elastic material may be rigid. The rigid portion of the shaft may be adjacent to the imaging device and the light source. In some aspects, the second size of the working channel may be a diameter ranging from about 1.5 mm to less than 2.5 mm. The opening of the working channel may be expandable up to a diameter of about 3 mm.

Further, according to some aspects, the shaft may include at least one articulation wire adjacent to the working channel. In some examples, the shaft may have a uniform outer diameter when the opening is not expanded, optionally wherein the uniform outer diameter ranges from about 3 mm to about 3.5 mm. A wall of the working channel adjacent to the imaging device and the light source may be rigid. The portion of the shaft including the elastic material may have a thickness ranging from about 0.1 mm to about 0.4 mm. The elastic material may be in the form of a patch integrated into the shaft at the distal tip.

According to some aspects, the medical device may include at least two instruments slidable along the working channel and through the opening, e.g., to expand the opening. For example, a combined cross-sectional size of the at least two instruments may be greater than the first size of the working channel. The medical device may include a handle coupled to the proximal portion of the shaft. The handle may include a port in communication with the working channel. The medical device may be useful to treat a subject, e.g., to collect biopsy samples of tissue from the subject.

The present disclosure also includes a medical device comprising a handle including a port; and a shaft coupled to the handle. The shaft may define a working channel in communication with the port and extending to an opening at a distal tip of the shaft, e.g., the opening being expandable. A distalmost face of the distal tip may include an imaging device and a light source proximate the opening. The working channel may have a first size at the opening and a second size proximal to the imaging device and the light source. A portion of the shaft around the opening and opposite the imaging device and the light source may include an elastic material. The opening may be expandable in a direction away from the imaging device and the light source.

In some examples, the elastic material comprises silicone or polyurethane. Additionally or alternatively, a portion of the shaft opposite the imaging device and the light source may be rigid. According to some aspects of the present disclosure, the second size of the working channel may be a diameter ranging from about 1.5 mm to less than 2.5 mm. Additionally or alternatively, the shaft may have a uniform outer diameter ranging from about 3 mm to about 3.5 mm when the opening is not expanded.

The present disclosure also includes method of treating a subject using a medical device as described above and/or elsewhere herein. For example, the method may include inserting a shaft of a medical device into a body lumen of the subject; and positioning a distal tip of the shaft proximate a target site. A distalmost face of the distal tip may include an imaging device, a light source, and an opening of a working channel of the shaft. At least a portion of the shaft around the opening may include an elastic material such that the opening is expandable in a direction away from the imaging device and the light source. The method may further include moving at least one instrument distally along the working channel and through the opening. The at least one instrument may have a cross-sectional size greater than a cross-sectional size of the opening such that the opening expands away from the imaging device and the light source as the at least one instrument passes through the opening. In some examples, the at least one instrument includes two instruments. For example, at least one of the two instruments may include forceps, e.g., useful for collecting one or more samples of tissue, such as in a biopsy procedure.

### BRIEF DESCRIPTION OF THE FIGURES

The accompanying drawings, which are incorporated in and constitute a part of this application, illustrate exemplary aspects that, together with the written descriptions, serve to explain the principles of this disclosure. Each figure depicts one or more exemplary aspects according to this disclosure, as follows:

FIG. 1 depicts an exemplary medical device according to some aspects of the present disclosure and includes a close-up cross-section of the distal tip.

FIG. 2A depict the distal end of the medical device of FIG. 1 in a first configuration with two instruments in a working channel proximal to the distal tip.

FIG. 2B depicts the distalmost face of the distal end of FIG. 2A.

FIG. 2C depicts a cross section of the distal end of FIG. 2A.

FIG. 3 depicts the distal end of the medical device of FIG. 1 with one of two instruments passing through the opening of the working channel.

FIG. 4A depicts the distal end of the medical device of FIG. 1 in a second configuration with two instruments passing through the opening of the working channel.

FIG. 4B depicts a cross section of the distal end of FIG. 4A.

### DETAILED DESCRIPTION

Particular aspects of the present disclosure are described in greater detail below. The terms and definitions provided herein control, if in conflict with terms and/or definitions incorporated by reference. The terms “proximal” and “distal” are used herein to refer to the relative positions of the components of exemplary medical devices. As used herein, “proximal” refers to a position relatively closer to the exterior of the body or closer to an operator using the medical device (see proximal “P” and distal “D” directional arrows in the figures). In contrast, “distal” refers to a position relatively further away from the operator using the medical device, or closer to the interior of the body.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of ±10% of the stated numeric value or range.

Although ureteroscopes are referenced herein for illustration purposes, it will be appreciated that the disclosure encompasses any suitable medical device configured to allow an operator to access and view internal body anatomy of a subject (e.g., patient) and/or to deliver medical instruments, such as, for example, biopsy forceps, graspers, baskets, snares, probes, scissors, retrieval devices, lasers, and other tools, into the subject's body. The medical devices herein may be inserted into a variety of body lumens and/or cavities, such as, for example, the urinary tract or gastrointestinal tract. It will be appreciated that, unless otherwise specified, bronchoscopes, duodenoscopes, endoscopes, gastroscopes, endoscopic ultrasonography (“EUS”) scopes, colonoscopes, ureteroscopes, bronchoscopes, laparoscopes, cystoscopes, aspiration scopes, sheaths, catheters, or any other suitable delivery device or medical device may be used in connection with the features described herein.

Aspects of the present disclosure provide medical devices with an enlarged working channel along a portion of a shaft and an expandable opening at the distal tip, e.g., to facilitate delivery of instruments for treatment of a subject. A medical device, such as an ureteroscope, may be inserted into a body lumen of a subject (e.g., into a bladder or kidney) in order to perform a medical procedure (e.g., a biopsy collection, e.g., to collect one or more samples of tissue). The distal tip of the medical device may include various electronic components, such as imaging devices and/or light sources. For example, the medical device may include one or more or light sources such as light emitting diodes (LEDs), optical fibers (e.g., plastic optical fibers (POFs), other light guides, or a combination thereof), imaging devices (e.g., cameras, other components having imagers, and/or other optical elements such as lenses), and/or other electronic components (e.g., capacitors, diodes, resistors, etc.). Electronic components of the assemblies herein may include electrical connections, and may also include various elements mounted, for example, or otherwise connected to, the electrical connections. According to some aspects herein, a distalmost face of the distal tip includes an imaging device and/or a light source proximate the opening of the working channel. The opening may be expandable, e.g., provided by elastic material of the shaft around at least part of the working channel and opening at the distal tip. In this way, an operator may navigate the medical device through tortuous anatomy while the distal tip has a reduced profile, and deliver multiple instruments through the working channel to a target site at the same time by temporary expansion of the working channel, without permanently enlarging the size of the distal tip. The following figures illustrate various features and examples of such medical devices.

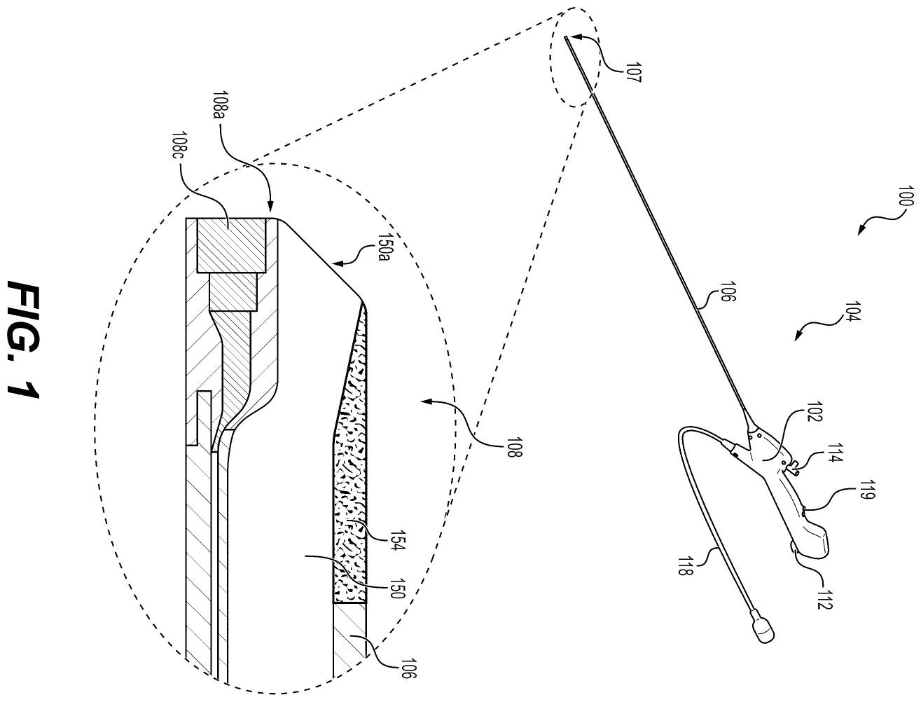

FIG. 1 depicts aspects of an exemplary medical device 100 according to the present disclosure. Medical device 100 includes a handle 102 and an insertion portion 104. Insertion portion 104 includes a shaft 106. Handle 102 may be configured for gripping and use by an operator. Shaft 106 includes a distal end 107, which includes a distal tip 108. Shaft 106 defines a working channel 150 extending longitudinally therethrough from a proximal portion of shaft 106 to an opening. For example, distal tip 108 may include a distal opening 150a of working channel 150. Distal opening 150a may be expandable, e.g., wherein at least a portion of shaft 106 around distal opening 150a comprises an elastic material that allows a portion of working channel 150 proximal to distal opening 150a to radially expand to accommodate one or more medical instruments passing therethrough.

At least a portion of shaft 106 may be flexible, for example, to facilitate navigation through a subject's anatomy. Shaft 106 may include one or more lumens or channels, the lumen(s)/channel(s) optionally containing wires (e.g., articulation members and/or wires for electric/electronic connection), tubes, or other features passing therethrough. For example, one or more channels 110, 111 may extend from a proximal end of shaft 106 to distal tip 108 to accommodate one or more articulation member(s). One or more channels 110, 111 and respective articulation members accommodated within channel(s) 110, 111, may be adjacent to working channel 150. The articulation member(s) may include wire(s) and/or cable(s) configured to articulate shaft 106 including distal tip 108 in one or more directions.

Handle 102 may include a mechanism to control articulation of medical device 100. For example, the mechanism may include an actuator such as lever 112 coupled to the articulation member(s). For example, lever 112 may be coupled to the articulation member(s) such that movement of lever 112 controls articulation and steering of shaft 106 including distal tip 108. It will be appreciated that any suitable actuator(s) may be used in addition or in place of lever 112, such as one or more knobs, buttons, sliders, switches, or joysticks. Handle 102 may include one or more locking mechanisms, for example, to lock a position of lever 112 and, thus, lock an orientation of an articulation portion of shaft 106 (e.g., locking an orientation of distal tip 108).

A port 114 of handle 102 may provide access to working channel 150 of shaft 106, e.g., port 114 being in fluid communication with working channel 150. An operator may insert one or more instruments (or other devices) into port 114 and may extend the instrument(s) distally through working channel 150 and through distal opening 150a. Optionally, handle 102 may include one or more ports in addition to port 114, each of which may receive one or more instruments and may be in fluid communication with working channel 150 so that the operator may extend the instrument(s) of the one or more additional portions through working channel 150 and distal opening 150a. In some examples, handle 102 may include a suction valve and/or other types of valves, such as, for example, fluid supply valves (e.g., air and/or water valves), and/or valves that perform a combination of functions.

Still referring to FIG. 1, an umbilicus 118 may extend from handle 102 and include or carry wires, cables, and/or conduits configured to provide, for example, power, signals, or fluids to and/or from handle 102. For example, umbilicus 118 may connect handle 102 to one or more user interfaces, monitors, controls units, displays, etc. As shown in FIG. 1, distal tip 108 includes a distalmost face 108a that defines opening 150a (e.g., distal opening 150a) of working channel 150. Opening 150a may be expandable to accommodate one or more instruments passed therethrough and extended distally beyond opening 150a.

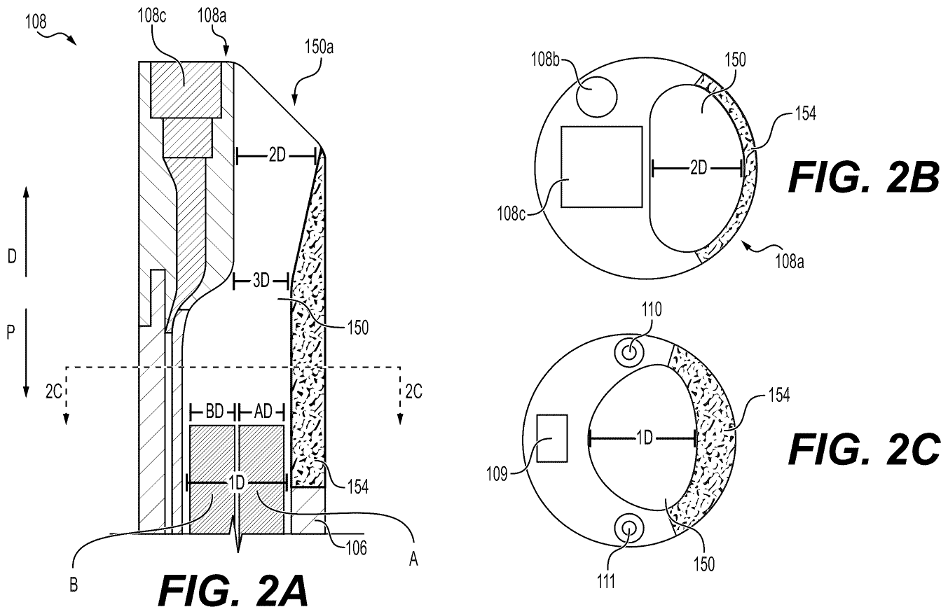

As mentioned above and at least shown in FIG. 2B, distal tip 108 may include one or more light sources 108b (e.g., LED(s)) and one or more imaging devices 108c (e.g., camera(s)). Although one light source 108b and one imaging device 108c are depicted in the figures, it will be appreciated that other combinations of light source(s) 108b and imaging device(s) 108c may be utilized. Light source 108b and imaging device 108c may be separate components or may be combined in single unit or device. Imaging device 108c may be configured to take video and/or still images. Imaging device 108c may provide a signal to a monitor or distal (e.g. connected via umbilicus 118), so that an operator may view an image provided by imaging device 108c while navigating medical device 100 through a body lumen of a subject.

An actuator of handle 102 operably coupled to imaging device 108c of insertion portion 104 may enable an operator to control imaging device 108c and capture images. For example, as shown in FIG. 1, handle 102 includes a button 119 to allow the operator to capture still images and/or video images via imaging device 108c. While button 119 is shown on a proximal portion of handle 102, button 119 (or other actuator) may be on another part of handle 102 accessible to the operator's hand during use. As depicted in FIG. 1, in some aspects, medical device 100 may be forward-facing. For example, features of distal tip 108 (e.g. opening 150a, light source(s) 108b, and/or imaging device 108c) may face the same direction, such as, for example, a distal direction (i.e. facing distally/forward of distalmost face 108a). In some examples, opening 150a may be angled relative to a longitudinal axis of shaft 106.

FIG. 2A shows a cross-sectional view of distal tip 108, FIG. 2B shows the distalmost face 108a (showing imaging device 108c and light source 108b at distalmost face 108a), and FIG. 2C shows a cross-sectional view proximal to imaging device 108c and light source 108b. FIG. 2C shows an electronic connection 109 to imaging device 108c and light source 108b (e.g., to provide power and control via umbilicus 118 of handle 102) and two channels 110, 111 for articulation members. While a single electronic connection 109 is shown (e.g., to both imaging device 108c and light source 108b), in other examples, the imaging device 108c and light source 108b may have separate electronic connections through shaft 106 and/or handle 102.

As shown at least in FIGS. 2A-2C, at least a portion of working channel 150 proximal to opening 150a (e.g., distal opening 150a) may have a size larger than the size of opening 150a. For example, working channel 150 may have a first size proximal to opening 150a (e.g., proximal to imaging device 108c and light source 108b at distalmost face 108a) that is greater than a second size at opening 150a. In examples in which working channel 150 has a circular cross-section, the first size may have a first diameter (labeled 1D in FIGS. 2A and 2C) and the second size may be a second diameter (labeled 2D in FIGS. 2A and 2B) smaller than the first diameter. Working channel 150 may include a transition portion between first diameter 1D and second diameter 2D, optionally a minimum diameter 3D of working channel 150 able to expand in size due to the elastic material 154 of shaft 106.

Working channel 150 may be sized and shaped to accommodate one or more instruments or other devices, e.g., two or more instruments simultaneously. In some examples, working channel 150 may have a uniform cross-sectional size from a proximal portion of shaft 106 until distal tip 108, e.g., where at distal tip 108 a portion of the cross-sectional size is taken up by electronic components at the distal tip 108 such as imaging device 108c and/or light source 108b. Thus, for example, working channel 150 may have a uniform diameter 1D until the transition portion between sizes 1D and 2D, and may have a smaller diameter 2D at opening 150a. Initially, second diameter 2D may accommodate only one instrument but may be capable of increasing in size due to an elastic material 154, e.g., by urging another instrument within working channel 150 distally toward and through opening 150a.

According to some aspects of the present disclosure, the first size (e.g.,

first diameter) of working channel 150 proximal to imaging device 108c and light source 108b ranges from about 2 mm to about 3 mm, and the second size (e.g., second diameter) of working channel 150 at opening 150a when not expanded is less than the first size. For example, the first size may be about 2.5 mm to about 3 mm, and the second size may be about 1.5 mm to less than 2.5 mm or less than 3 mm. In some aspects, shaft 106 has a uniform outer diameter when opening 150a is not expanded, e.g., a diameter ranging from about 3 mm to about 3.5 mm, or from about 3.1 mm to about 3.3 mm.

As mentioned above, distal tip 108 may include a region around opening 150a comprising an elastic material 154. Elastic material 154 may define at least a portion of working channel 150 and opening 150a. Elastic material 154 may take the form of a patch or lip of distal end 107 of shaft 106, e.g., integrated into the remaining/surrounding material forming shaft 106 (which may be rigid, semi-rigid, or flexible but not elastic). Elastic material 154 may be present in a single, continuous piece or may be incorporated as multiple regions within a non-elastic material that allows the portion of shaft 106 that includes elastic material 154 to expand radially outward.

The thickness of elastic material 154 may be uniform, or may taper, e.g., in the proximal to distal direction as shown in FIG. 1. The thickness of elastic material 154 may range from about 0.1 mm to about 0.4 mm, e.g., from about 0.2 mm to about 0.3 mm. Exemplary elastic materials suitable for the present disclosure include, but not limited to, flexible, elastic polymers such as silicone and polyurethane. Elastic material 154 may form at least a portion of a perimeter around opening 150a of distal tip 108. In some aspects, the entirety of distal tip 108 may be formed from or otherwise comprise an elastic material. In such cases, the side (e.g., a portion) of shaft 106 opposite opening 150a and/or adjacent to imaging device 108c and/or light source 108b may be rigid (e.g., a rigid portion) due to proximity to rigid electronic components such as imaging device 108c and/or light source 108b such that opening 150a expands in a direction away from imaging device 108c and light source 108b.

FIGS. 2A-2C, 3, and 4A-4B illustrate expansion of opening 150a (e.g., distal opening 150a) of working channel 150 due to passage of multiple instruments therethrough. For example, two instruments (e.g., instrument A and instrument B) may be inserted through port 114 (or different ports in communication with working channel 150), extended (e.g., moved) through working channel 150, and extended (e.g., moved) through and distally relative to opening 150a. It should be understood that while the figures show two instruments, one instrument or more than two instruments (e.g., at least two instruments) may be slidable along working channel 150 and/or may be advanced along working channel 150, e.g., within size constraints of working channel 150 and shaft 106. Each instrument A, B may include a diameter DA and DB, respectively. It should be understood that although diameters DA and DB are illustrated in the figures as being approximately the same diameter, diameters DA and DB may be any diameter so long as the combined size (for example, a combined cross-sectional size) DA and DB is less than the cross-section size of working channel 150, diameter D1. For example, in some aspects, DA may be twice the diameter of DB. In another example, there may only be one instrument (such as instrument A) where diameter DA is less than 1D but greater than the size of opening 150a (diameter 2D).

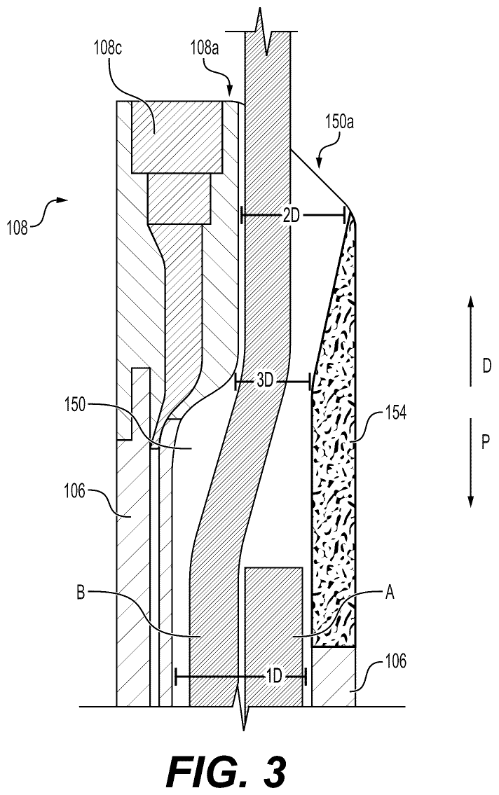

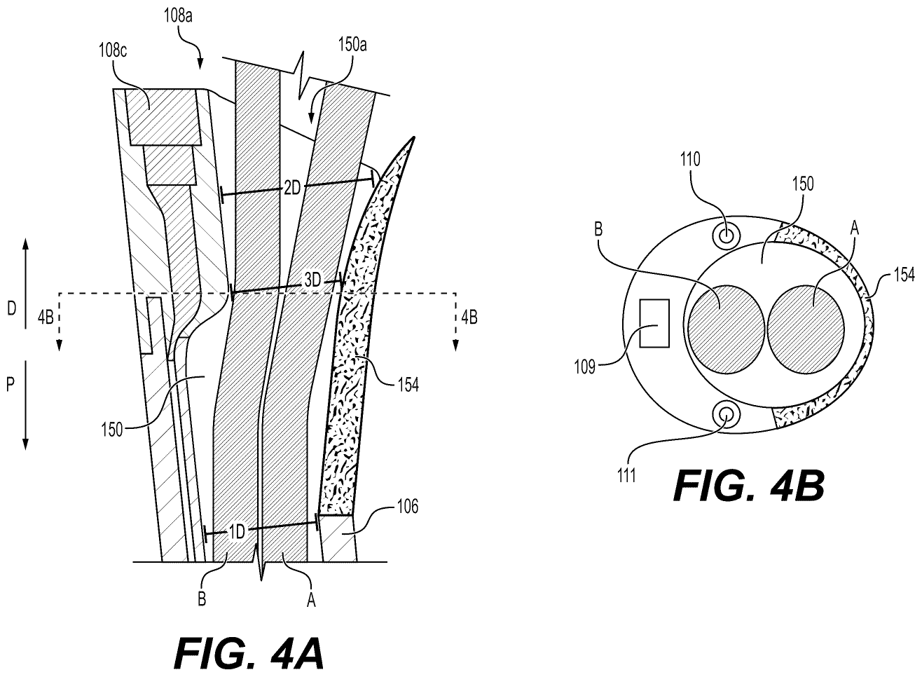

Opening 150a may expand when the one or more instruments A, B have a size greater than opening 150a and are passed therethrough. For example, as shown in FIG. 3, instrument B is extended distally beyond diameter 3D and opening 150a (e.g., diameter 2D), however diameter DB is lesser than diameter D3, thus instrument B is able to pass through opening 150a without expanding elastic material 154. FIGS. 4A and 4B illustrate instruments A and B both extending through opening 150a at the same time. Because their combined size (diameters DA and DB) is greater than diameter D3, instruments A, B, push against the wall of working channel 150 causing elastic material 154 to expand in a direction away from imaging device 108c and light source 108b. When diameter DA, diameter DB, and/or the combined size of diameters DA and DB is greater than the size of working channel 150 in distal tip 108 (e.g., diameter 3D), the instrument(s) A, B may cause the size of working channel 150 at and proximate opening 150a to expand radially outward relative to the longitudinal axis of medical device 100, that is, away from imaging device 108c and light source 108b. In this expanded configuration, diameter 2D and/or diameter 3D may expand relative to their original sizes, and optionally may be greater than the size of the proximal portion of working channel 150 (e.g., diameter 1D). Once instrument(s) A, B are retracted, the elasticity of elastic material 154 may allow shaft 106 to return to its original size, e.g., with a reduced profile that facilitates navigation through body lumens. It should be understood that the expansion of working channel 150 and opening 150a in response to one or more instruments A, B being extended therethrough would allow the operator to collect one or more samples of tissue (e.g., one or more biopsy samples) with instruments A, B where each instrument may be configured to receive/capture one or more samples of tissue. For example, at least one or both instruments A, B, may include a forceps, grasper, basket, or other retrieval device, etc.

Due to the elastic, flexible properties of distal tip 108, opening 150a is able to be sized smaller than otherwise necessary to accommodate instruments able to traverse working channel 150. A relatively smaller opening 150a may allow for distalmost face 108a to include various electronic and/or other components, without increasing the overall profile of shaft 106.

While principles of the disclosure are described herein with reference to illustrative aspects for particular medical uses and procedures, the disclosure is not limited thereto. Each of the aspects disclosed herein may include one or more of the features described in connection with any of the other aspects. Those having ordinary skill in the art and access to the teachings provided herein will recognize additional modifications, applications, aspects, and substitution of equivalents all fall in the scope of the aspects described herein. Accordingly, the disclosure is not to be considered as limited by the foregoing description.

What is claimed is:

1. A medical device comprising:
a shaft defining a working channel extending from a proximal portion of the shaft to an opening at a distal tip of the shaft, a distalmost face of the distal tip including an imaging device and a light source proximate the opening;
wherein the working channel has a first size at the opening and a second size proximal to the imaging device and the light source that is greater than the first size; and
wherein at least a portion of the shaft around the opening comprises an elastic material such that the opening is expandable in a direction away from the imaging device and the light source.

2. The medical device of claim 1, wherein the elastic material comprises a flexible, elastic polymer.

3. The medical device of claim 2, wherein the elastic material comprises silicone or polyurethane.

4. The medical device of claim 1, wherein a portion of the shaft opposite the elastic material is rigid, the rigid portion of the shaft being adjacent to the imaging device and the light source.

5. The medical device of claim 1, wherein the second size is a diameter ranging from about 1.5 mm to less than 2.5 mm.

6. The medical device of claim 1, wherein the opening is expandable up to a diameter of about 3 mm.

7. The medical device of claim 1, wherein the shaft includes at least one articulation wire adjacent to the working channel.

8. The medical device of claim 1, wherein the shaft has a uniform outer diameter when the opening is not expanded.

9. The medical device of claim 1, wherein a wall of the working channel adjacent to the imaging device and the light source is rigid.

10. The medical device of claim 1, wherein the portion of the shaft comprising the elastic material has a thickness ranging from about 0.1 mm to about 0.4 mm.

11. The medical device of claim 1, wherein the elastic material is in the form of a patch integrated into the shaft at the distal tip.

12. The medical device of claim 1, further comprising at least two instruments slidable along the working channel and through the opening, wherein a combined cross-sectional size of the at least two instruments is greater than the first size.

13. The medical device of claim 1, further comprising a handle coupled to the proximal portion of the shaft, wherein the handle includes a port in communication with the working channel.

14. A medical device comprising:
a handle including a port; and
a shaft coupled to the handle, the shaft defining a working channel in communication with the port and extending to an opening at a distal tip of the shaft, wherein a distalmost face of the distal tip includes an imaging device and a light source proximate the opening;
wherein the working channel has a first size at the opening and a second size proximal to the imaging device and the light source; and
wherein a portion of the shaft around the opening and opposite the imaging device and the light source comprises an elastic material, the opening being expandable in a direction away from the imaging device and the light source.

15. The medical device of claim 14, wherein the elastic material comprises silicone or polyurethane.

16. The medical device of claim 14, wherein a portion of the shaft opposite the imaging device and the light source is rigid.

17. The medical device of claim 14, wherein the second size is a diameter ranging from about 1.5 mm to less than 2.5 mm.

18. The medical device of claim 17, wherein the shaft has a uniform outer diameter ranging from about 3 mm to about 3.5 mm when the opening is not expanded.

19. A method of treating a subject, the method comprising:
inserting a shaft of a medical device into a body lumen of the subject;
positioning a distal tip of the shaft proximate a target site, wherein a distalmost face of the distal tip includes an imaging device, a light source, and an opening of a working channel of the shaft, at least a portion of the shaft around the opening comprising an elastic material such that the opening is expandable in a direction away from the imaging device and the light source; and
moving at least one instrument distally along the working channel and through the opening, wherein the at least one instrument has a cross-sectional size greater than a cross-sectional size of the opening, such that the opening expands away from the imaging device and the light source as the at least one instrument passes through the opening.

20. The method of claim 19, wherein the at least one instrument includes two instruments, at least one of the two instruments including a forceps.
